(19) United States
(12) Patent Application Publication
(10) Pub. No.: US 2002/0123086 A1
(43) Pub. Date: Sep. 5, 2002

(54) Method to distinguish whether an event sequence is a memory driven event sequence or is not a memory driven event sequence

(76) Inventors: Rudolf Rigler, St. Sulpice (SE); Lars Edman, Stockholm (SE)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/020,888

(22) Filed: Dec. 19, 2001

Related U.S. Application Data
(60) Provisional application No. 60/257,146, filed on Dec. 22, 2000.

(51) Int. Cl. C12Q 1/00

(52) U.S. Cl. 435/8; 436/172

(57) ABSTRACT

The present invention concerns a method to distinguish between memory-driven and non-memory driven processes by the use of higher order correlation functions. In particular, it concerns means to investigate memory driven processes in enzymatic catalysis, and especially in single molecule sequencing reactions.

### DESCRIPTION

The present invention concerns a method to distinguish, whether an event sequence is a memory driven event sequence or is not a memory driven event sequence. In particular, the present invention concerns means to investigate memory driven processes in enzymatic catalysis, and especially in single molecule sequencing reactions.

Processes that happen independently of each other on the molecular level do not show any signs of memory. In other words, the future state of the system does not depend on the previous states of the system. In contrast, if individual molecules, and in particular individual enzymes or substrates are involved, it is likely that the previous state of the system influences future states of the system, i.e. that the system has memory. This has previously been shown for cholesterol oxidase (Lu, 1998), where it is believed that the system does not just cycle between the two spectroscopically observable states, but instead goes through a whole cycle of intermediate states between subsequent steps of actual catalysis. As a result, the catalytic machinery is a strongly memory dependent system.

In mathematical terms, memory processes reflect a divergence from the Markov assumption. Define {Xₜ} as a stochastic process. {Xₜ} is binary in the sense that its event room W contains only two elements: W={0, 1}. {Xₜ} is stationary in the sense that its expectation value E{Xₜ}=m, where 0<m<1 is a constant (not time dependent). If dt is considered a very small time interval, the two possible values Xₜ=0 and Xₜ=1 represent the possibility that an event has failed to occur (Xₜ=O) or has occured (Xₜ=1). This event can be the emission of a photon from a molecule, the binding or release of a substrate from an enzyme, if this can be monitored, or any other event, in particular any event at the molecular level.

The Markov assumption can then formally be written:

P(Xₜ_{_{N}}|Xₜ_{_{N-1}};Xₜ_{_{N-2}}; . . . ;Xₜ_{₀})=P(Xₜ_{_{N}}|Xₜ_{_{N-1}}), t₀<t₁<. . . <t_{N}.

If Eq. 1 is valid, we also have the following weaker but still valid statement:

P(Xₜ_{_{N|X}}ₜ_{_{N-Z}})=P(Xₜ_{_{N}}|Xₜ_{_{N-t}}).

The non-Markovian function (NMF) for the observed process {Xₜ} is can be defined as:

NMF(t_{N}−t_{N -1}, t _{N-1}−t_{N-2})=P(Xₜ_{_{N}}|Xₜ_{_{N-1}}; Xₜ_{_{N-2}})−P(Xₜ_{_{N}}|Xₜ_{_{N-1}})

Because {Xₜ} is a stationary process, NMF has only two arguments (instead of three in the more general case if {Xₜ} is not stationary) that equal the time differences between the three observation times.

It is the task of the present invention to provide a method to distinguish, whether an event sequence is a memory driven event sequence or is not a memory driven event sequence on a time scale T₁ to T₂, where T₁<T₂ are arbitrary times.

This task is solved by a the demonstratation, that memory driven event sequences can be discriminated against non-memory driven event sequences on the basis of their first and second order autocorrelation functions, that are experimentally measurable quantities. Specifically, a method is disclosed, wherein

(a) the first order autocorrelation function G(τ) of the event sequence is calculated,

(b) the second order autocorrelation function G(τ₁, τ₂) of the event sequence is calculated,

(c) it is decided that the event sequence is a memory driven event sequence on the time scale T₁ to T₂,

if the second order autocorrelation function of the event sequence can be expressed within experimental error as the product of first order autocorrelation functions, i.e. G(τ₁,τ₂)=G(τ₁)*G(τ₂) for T₁<τ₁, τ₂<T₂, and

(d) it is decided that the event sequence is not a memory driven event sequence on the time scale T₁ to T₂,

if the second order autocorrelation function of the event sequence cannot be expressed within experimental error as the product of first order autocorrelation functions, i.e. G(τ₁,τ₂)≠G(τ₁)*G(τ₂) for T₁<τ₁, τ₂<T₂.

An understanding of the method is best gained from a definition of the first and second order autocorrelation functions for a series of events {Xₜ}. Let E(.) denote the expectation value of a random variable. Set t_{N}−t_{N-1}=τ₁ and t_{N-1}−t_{N-2}=τ₂. The time τ₂ is, hence, the time in addition to the time τ₁ from the reference time t_{N}, which we set arbitrarily to zero because the process is stationary. Probabilities are expressed with the usual symbol P, and the bar (|) denotes conditional probabilities. As usual, all conditions are denoted on the right side of the bar. For example

P(X₀=1|Xᵣ=1)

denotes the probability, that X at time t=0 is 1, provided it was also 1 a time τ ago.

By definition, the first order autocorrelation function, also referred to as first order correlation function for brevity, is:

$$\begin{matrix}G ( τ ) ≡ \frac{E ( {X}_{0} {X}_{τ} )}{E ( {X}_{0} ) E ( {X}_{τ} )} = \frac{{\sum }_{i = 0}^{1} {\sum }_{i = 0}^{1} ijP ( {X}_{0} = i ; {X}_{τ} = j )}{{[ {\sum }_{i = 0}^{1} i P ( {X}_{0} = i ) ]}^{2}} \\ = \frac{{\sum }_{i = 0}^{1} {\sum }_{j = 0}^{1} ij P ( {X}_{0} = i \vert {X}_{τ} = j ) P ( {X}_{τ} = j )}{{[ {\sum }_{i = 0}^{1} i P ( {X}_{0} = j ) ]}^{2}} \\ = \frac{P ( {X}_{0} = 1 \vert {X}_{τ} = 1 )}{P ( {X}_{0} = 1 )}\end{matrix}$$

Similarly, the second order autocorrelation function, also referred to as second order correlation function for brevity, is defined as:

$$\begin{matrix}G ( {τ}_{1} , {τ}_{2} ) ≡ \frac{E ( {X}_{0} {X}_{{τ}_{1}} {X}_{{τ}_{1} + {τ}_{2}} )}{E ( {X}_{0} ) E ( {X}_{{τ}_{1}} ) E ( {X}_{{τ}_{1} + {τ}_{2}} )} = \frac{{\sum }_{i = 0}^{1} {\sum }_{j = 0}^{1} {\sum }_{k = 0}^{1} ijkP ( {X}_{0} = i ; {X}_{{τ}_{1}} = j ; {X}_{{τ}_{1} + {τ}_{2}} = k )}{{[ {\sum }_{i = 0}^{1} i P ( {X}_{0} = i ) ]}^{3}} \\ = \frac{{\sum }_{i = 0}^{1} {\sum }_{j = 0}^{1} {\sum }_{k = 0}^{1} ij k P ( {X}_{0} = i \vert {X}_{{τ}_{1}} = j ; {X}_{{τ}_{1} + {τ}_{2}} = k ) P ( {X}_{{τ}_{1}} = j ; {X}_{{τ}_{1} + {τ}_{2}} = k )}{{[ {\sum }_{i = 0}^{1} i P ( {X}_{0} = i ) ]}^{3}} \\ = \frac{\begin{matrix}{\sum }_{i = 0}^{1} {\sum }_{j = 0}^{1} {\sum }_{k = 0}^{1} ij kP ( {X}_{0} = i \vert {X}_{{τ}_{1}} = j ; {X}_{{τ}_{1} + {τ}_{2}} = k ) \\ P ( {X}_{{τ}_{1}} = j \vert {X}_{{τ}_{1} + {τ}_{2}} = k ) P ( {X}_{{τ}_{1} + {τ}_{2}} = k )\end{matrix}}{{[ {\sum }_{i = 0}^{1} i P ( {X}_{0} = i ) ]}^{3}} \\ = \frac{P ( {X}_{0} = 1 \vert {X}_{{τ}_{1}} = 1 ; {X}_{{τ}_{1} + {τ}_{2}} = 1 ) P ( {X}_{{τ}_{1}} = 1 \vert {X}_{{τ}_{1} + {τ}_{2}} = 1 )}{{( P ( {X}_{0} = 1 ) )}^{2}}\end{matrix}$$

In the case of a non-memory driven process,

P(X₀=1|X_{τ1}=1; X_{τ1+τ2}=1)=P(X₀=1|X_{τ1}=1),

because in a process without memory, the time τ₁+τ₂ ago cannot have an effect, provided the event at time τ₁ ago is known. In this case, the expression for the second order correlation function can be expressed simply as a product of first order correlation functions, i.e. G(τ₁,τ₂)=G(τ₁)*G(τ₂) for T₁<τ₁, τ₂<T₂, where T₁ and T₂ delimit the time range, for which the process has no memory.

For systems that do have memory, the degree of memory can be expressed in terms of the non-Markovian function as explained in the introduction. The non-Markovian function (NMF) can be expressed in terms of first and second order autocorrelation functions. Using the definition of the NMF and the expressions for the first and second order autocorrelation functions derived above, it can easily be shown that

$$NMF ( {τ}_{1} , {τ}_{2} ) = {p}_{f} ( \frac{G ( {τ}_{1} , {τ}_{2} )}{G ( {τ}_{2} )} - G ( {τ}_{1} ) ) ,$$

where ρ_{f}=P(X_{O}=1) is the probability of the event X at a particular time.

The formula is best understood from a consideration of limiting cases. Assume that the process has no memory. In this case, for arbitrary τ₁ and τ₂, G(τ₁)*G(τ₂)=G(τ₁,τ₂), and correspodingly, NMF(τ₁, τ₂)=0. This is as expected from the definition of the NMF, that should be 0 for memory free processes. Conversely, if the process does have memory, and G(τ₁)*G(τ₂)≠G(τ₁,τ₂), NMF(τ₁,τ₂) is a non-trivial function of the two real variables τ₁ and τ₂. In this case, the two-dimensional plot of NMF as a function of τ₁ and τ₂ is the non-trivial memory landscape (ML) of the process under observation.

The decribed method is only valid, if the bin size in time used for recording the autocorrelation functions is small enough so that only zero or one event is registered per bin. It means that no two-state emission dynamics can be monitored on faster time ranges than the inverse of the bin size (50 s⁻¹in the example). However, for two-state dynamics that have larger characteristic times than the inverse of the bin-size, the NMF correctly displays deviations from Markovian dynamics and yields a valid memory landscape.

Autocorrelation functions can be recorded in many circumstances. However, recording is most convenient by optical methods, if the molecular events under investigation are associated with a change of the spectroscopic or fluorescence properties of the sample. If a change of fluorescence is involved, standard confocal microscopy (Eigen, 1994; Edman, 1999) can be used for fluorescence detection. This is further illustrated in Example 1 for the oxidation of dihydrorhodamine 6G by horseradish peroxidase. In all experimental setups, the temporal resolution of memory effects depends on the temporal resolution for the autocorrelation functions.

When a sequence of fluorescence events is recorded, the method according to the invention can be used to discriminate an event sequence from a single molecule against an event sequence from background processes or noise. It is decided that the event sequence is due to a single molecule, if it is a memory driven event sequence, and that the event sequence is due to background processes or noise, if it is a non-memory driven event sequence.

The appearance of memory effects (i.e. non-zero memory landscapes) in the behaviour of single molecules is expected both on theroretical and on experimental grounds. It can for example be seen from theoretical predictions of the kinetics of single enzyme systems (Ryde-Petterson, 1989; Jackson, 1989). These predictions are based on the idea that the dynamic process of a single enzyme performing catalysis is not an equilibrium process. This is so, because there is a continuous flow through the system (observe that the system is defined as the single enzyme molecule and all substrate as well as product molecules interacting with the single enzyme). The flow consists of substrate molecules that enter the system irreversibly leave the system as products. If a kinetic model of such a non-equilibrium system is made with at least one intermediate state and one enzyme-product state, the eigenvalues to the corresponding rate matrix may be complex, leading to sine and cosine solutions (Ryde-Petterson, 1989; Jackson, 1989). Such oscillations are clearly non-Markovian and hence can be observed as non-trivial memory landscapes of the NMF.

The appearance of memory effects in enzymes is expected also on experimental grounds. Streched exponential decay has been observed in fluorescence decay (FD) measurements. It is known from theoretical work by Palmer and coworkers (Palmer, 1984) that such streched exponential processes can be observed in complex systems where the transition from one state to the other depends on a number of subprocesses, provided the subprocesses must always be completed before the main process changes its state. It is strongly expected that systems with many internal states will display complex memory effects.

The time-scale of memory effects in individual molecules is thus expected to vary widely. Fluorescence decay processes typically happen on a time-scale of ns or even shorter, whereas for chemical reactions effects in the ms to s timescale are more typical. The current invention can be used for any of these timescales, provided the measurement equipment allows sufficient temporal resolution.

In contrast to events from single molecules, many background processes that originate from independent “background” events and also many types of noise do not show memory effects. As a consequence, the method according to the invention can be used to discriminate an event sequence from a single molecule against an event sequence from background processes or noise.

The method can be used particularly well in single molecule sequencing reactions. In single molecule sequencing (Dörre, 1997), nucleotides are processively cleaved from the DNA molecule for sequencing. It is expected that the polymerase proceeds smoothly, releasing nucleotides in roughly regular time intervals. An analysis of nucleotide release (or detection) events should therefore reveal a prominent memory landscape. Conversely, if contaminating nucleotides are present, their appearance in an observation element of the single molecule sequencing unit will be a random process not governed by memory effects. Accordingly, in single molecule sequencing, it is decided that

(a) the fluorescence events observed in a confocal microscope are due to nuclease-liberated nucleotides if the sequence of fluorescence events is a memory driven sequence of events and

(b) the fluorescence events observed in a confocal microscope are due to contaminating nucleotides or other background signals, if the sequence of fluorescence events is not a memory driven sequence of events.

It is clear that the step from first to second order correlation functions can be generalised to lead from second order to third order correlation functions and so on. Thus, the “memory” of the “memory” can be investigated.

According to a further aspect of the present invention a method is provided for analyzing of catalytic complexes, wherein the method is characterized in that

(a) it is decided that the fluorescence events observed in a confocal microscope are due to characteristics of the catalytic complex if the sequence of fluorescence events is a memory driven sequence of events and

(b) it is decided that the fluorescence events observed in a confocal microscope are due to contaminating nucleotides or other background signals, if the sequence of fluorescence events is not a memory driven sequence of events.

The catalytic complex may comprise for example a catalyst, a substrate being converted to a product and optionally a cosubstrate.

Preferably the catalyst is selected from biomolecules, e.g. enzymes, inorganic molecules and organic molecules.

In general the method according to the present invention may be performed for analysing oscillatory processes.

### EXAMPLE 1

As an example for the detection of non-Markovian behaviour of single molecules, the measurement and calculation of the NMF for a single molecule of horseradish peroxidase will be described.

Horseradish peroxidase is a 44-kDa heme protein and is an effective catalyst of the decomposition of hydrogen peroxide (H₂O₂) in the presence of hydrogen donors (Willsäatter, 1923). Dihydrorhodamine 6G was chosen as substrate, so that the catalysis reaction can be described as:

RH₂+H₂O₂→R+2H₂O,

where RH₂ represents dihydrorhodamine 6G and R represent rhodamine 6G.

The advantage of this system is that the enzyme, substrate and enzyme-substrate complex are non-fluorescent. In contrast, the enzyme-product complex (EP) is fluorescent and is formed as a result of the substrate being oxidized while still bound to the enzyme. Thus, the catalyis reaction can be monitored by existing experimental methods based on confocal fluorescence spectroscopy (Rigler, 1992; Mets, 1994).

The confocal microscope that is used for the present set of experiments has been described before (Edman, 1999). The biotinylated enzyme is bound to a streptavidinized glass coverslip surface. The substrate solution is applied as a “hanging droplet” Experiments were carried out at a substrate (dihydrorhodamine 6G) concentration of 130 nM, H₂O₂ concentration of 120 mM, in 100 mM potassium phosphate buffer at pH 7.0.

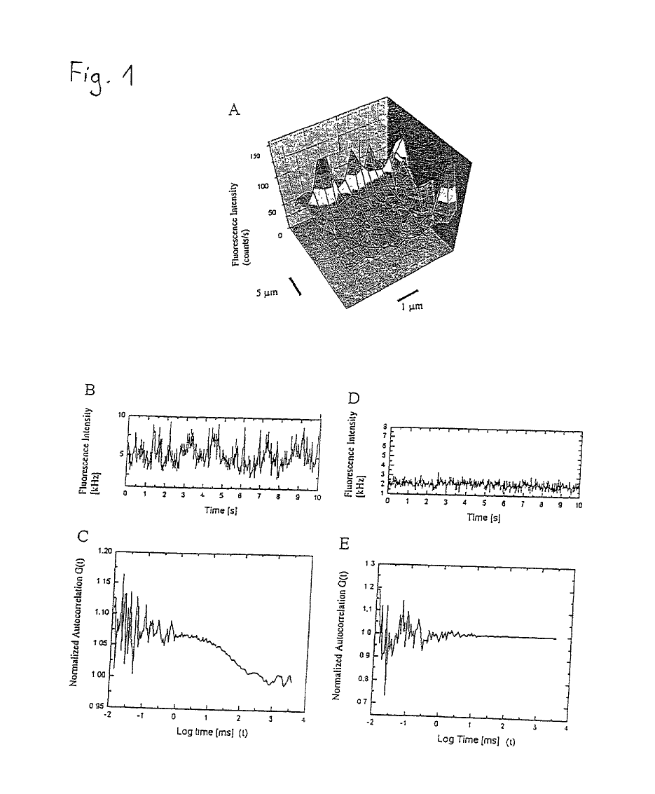

To find a single-enzyme molecule, a scanning procedure is conducted in which the open volume element from where the fluorescence is detected is moved in a direction parallel to the coverslip surface until a single-enzyme molecule is detected (FIG. 1A). The signature of a single enzyme molecule is that of fluctuations in the fluorescence intensity traces combined with a clear signal in the autocorrelation function of the intensity fluctuations (FIGS. 1B and C).

When no enzyme is present, the fluorescence intensity traces show only background signal, and the fluorescence intensity autocorrelation function is flat (FIGS. 1 D and E). Another control experiment shows a blank in the absence of H₂O₂, but with all other ingredients present (not shown). It is therefore concluded that fluctuations in the presence of enzyme must originate from the enzyme interaction with the substrate.

The finding that the average fluorescence intensity is continuously increasing inside the sample solution when enzyme is bound to the glass surface, but not otherwise (when no enzyme is present), indicates that the surface bound enzymes are active.

Additional control assays done in the bulk indicate that the average substrate turnover rate is 34 s⁻¹, which is roughly in line with the average of the observed substrate turnover rates, and product dissociation rates from single enzyme molecules.

The above facts combined make us conclude that single enzymes that catalyse the conversion from substrate to product are observed. Thus, first and second order autocorrelation functions G(τ₁) and G(τ₁, τ₂) could be recorded and the NMF could be calculated.

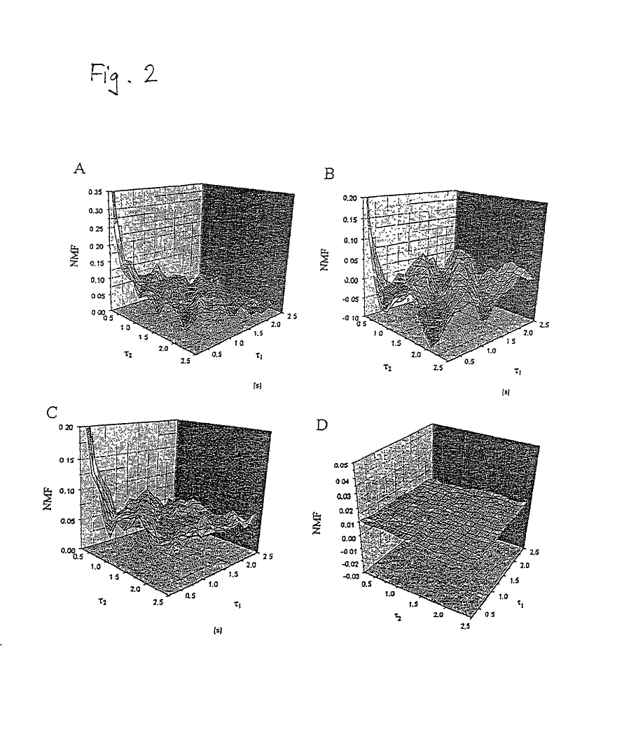

In FIGS. 2 A-C, the ML are shown for three horseradish peroxidase molecules observed for 110 s. Many molecules have been observed; FIG. 2 shows examples. Indeed, the ML show non-Markovian behavior on the 2.5-s time scale. Apart from a clear memory at shorter times (<100 ms), there are structures in the memory landscape for all molecules in the range of seconds. It is also evident that, even though the 110-s ML are not identical, they all have a characteristic pattern with elongated valleys and peaks diagonally in the ML. A peak or a valley in which NMF≠0 indicates that the knowledge of the spectroscopic state at the additional historical time τ₂ influences the state probability at time 0. In contrast to the ML generated from the data from the single enzymes performing catalysis, ML from data taken in the absence of enzyme (but everything else held constant) show a flat unstructured landscape with values close to zero (FIG. 2D).

General remark:

A method according to the present invention may be performed e. g. on the basis of the fluorescence correlation spectroscopy (FCS) technology and with the equipment described in EP 0 679 251 B1 or DE 195 08 366 C2 which are incorporated into the present application by reference.

It is to be noted that the correlation functions, particularly the autocorrelation functions of first, second or higher order calculated from measurement data, particularly fluorescence-measurement data, are one possibility of representation. The correlation functions may be transformed into the corresponding power spectrum (Wiener-Khinchin theorem). Alternatively it is possible to calculate or derive a power spectrum directly from fluorescence measurement data. The power spectrum contains the information of the corresponding correlation function. Therefore, the power spectrum of the corresponding order may be the basis for distinguishing, whether an event sequence is a memory driven event sequence or is not a memory driven event sequence, according to the present invention. It is also possible to first calculate the power spectrum and then to transform the power spectrum into the corresponding correlation function. Further, the power spectrum, particularly a higher order power spectrum, may be directly evaluated to analyze event sequences, e.g. of oscillatory phenomena and multiple processes.

References:

1. Döorre, K. et al. (1997) Bioimaging 5, 139-152.

2. Edman, L., Földes-Papp, Z., Wennmalm, S. & Rigler, R. (1999) Chem. Phys. 247, 11-22.

3. Eigen, M., Rigler, R. (1994) Proc. Nat. Acad. Sci. 91, 5740-5747.

4. Elson, E., Magde, D. (1974) Biopolymers 13, 1-27.

5. Jackson, E. A. (1989) Perspectives of Nonlinear Dynamics (Cambridge Univ. Press, Cambridge, U.K.), Vol. 1.

6. Lu, H. P., Xun, L. & Xie, X. S. (1998) Science 282, 1877-1882.

7. Mets, Ü & Rigler, R. (1994) J. Fluoresc. 4,259-264.

8. Palmer, R. G., Stein, D. L., Abrahams, E. & Anderson, P. W. (1984) Phys. Rev. Lett. 54,958-961

9. Qian, H. (1990) Biophys. Chem. 38, 49-57.

10. Rigler, R. & Mets, Ü. (1992) SPIE Laser Spectrosc. Biomol. 1921, 239-248.

11. Ryde-Pettersson, U. (1989) Eur. J. Biochem. 186, 145-148.

12. Willsätter, R. & Pollinger, A. (1923) A. Liebigs Ann. 430, 269-319.

FIG. 1: (A) A surface scan provides a fluorescence image of single enzyme molecules. (B) and (C) The signature of a single enzyme performing catalysis is that of fluctuations in the intensity trace (B) combined with a clear signal in the autocorrelation function (C). (D and E) A control experiment in which no enzyme is present (but with everything else held constant) shows only background signal in the intensity trace (D) and no autocorrelation signal (E).

FIG. 2: Memory landscapes (ML) are shown for three molecules observed for 110 s in A, B and C. The relative errors were calculated to be less then ±3%, ±4.5% and ±3% for all points in the memory landscape of A, B and C, respectively. D shows a memory landscape generated from measurement data generated for the case when no enzyme is present.

1. Method to distinguish, whether an event sequence is a memory driven event sequence or is not a memory driven event sequence on a time scale T₁ to T₂, where T₁ <T₂ are arbitrary times, characterized in that
(a) the first order autocorrelation function G(T) of the event sequence is calculated,
(b) the second order autocorrelation function G(τ₁, τ₂) of the event sequence is calculated,
(c) it is decided that the event sequence is a memory driven event sequence on the time scale T₁ to T₂,
if the second order autocorrelation function of the event sequence can be expressed within experimental error as the product of first order autocorrelation functions, i.e. G(τ₁, τ₂)=G(τ₁)*G(τ₂) for T₁<τ₁, τ₂<T₂, and
(d) it is decided that the event sequence is not a memory driven event sequence on the time scale T₁ to T₂,
if the second order autocorrelation function of the event sequence cannot be expressed within experimental error as the product of first order autocorrelation functions, i.e. G(τ₁, τ₂)≠G(τ₁)*G(τ₂) for T₁<τ₁, τ₁<T₂.

2. Method according to claim 1, characterized in that
(a) the first order autocorrelation function G(τ) of the event sequence is calculated as:
$$G ( τ ) ≡ \frac{E ( {X}_{0} {X}_{τ} )}{E ( {X}_{0} ) E ( {X}_{τ} )}$$
where X is the random variable that describes the event and E(.) denotes the expectation value,
(b) the second order autocorrelation function G(τ₁, τ₂) of the event sequence is calculated as:
$$G ( {τ}_{1} , {τ}_{2} ) ≡ \frac{E ( {X}_{0} {X}_{{τ}_{1}} {X}_{{τ}_{1} + {τ}_{2}} )}{E ( {X}_{0} ) E ( {X}_{{τ}_{1}} ) E ( {X}_{{τ}_{1} + {τ}_{2}} )}$$
where X is the random variable that describes the event and E(.) denotes the expectation value,

3. Method according to claim 1, characterised in that the degree of memory of the system is quantified by the non-Markovian function NMF calculated according to:
$$NMF ( {τ}_{1} , {τ}_{2} ) = {p}_{f} ( \frac{G ( {τ}_{1} , {τ}_{2} )}{G ( {τ}_{2} )} - G ( {τ}_{1} ) ) ,$$
where ρ_{f} is the probability of the event X at a particular time.

4. Method according to claim 1, characterized in that the event sequence is a sequence of fluorescence events observed in a confocal microscope.

5. Method according to claim 4, to discriminate an event sequence from a single molecule against an event sequence from background processes or noise, characterized in that
(a) it is decided that the event sequence is due to a single molecule, if it is a memory driven event sequence,
(b) it is decided that the event sequence is due to background processes or noise, if it is a non-memory driven event sequence.

6. Method according to claim 5, for single molecule sequencing, characterized in that
(a) it is decided that the fluorescence events observed are due to nuclease-liberated nucleotides if the sequence of fluorescence events is a memory driven sequence of events and
(b) it is decided that the fluorescence events observed are due to contaminating nucleotides or other background signals, if the sequence of fluorescence events is not a memory driven sequence of events.

7. Method according to claim 6, characterized in that the fluorescence events are observed in a confocal microscope.

8. Method according to claim 6 or 7 for analyzing of catalytic complexes, characterized in that
(a) it is decided that the fluorescence events observed are due to characteristics of the catalytic complex if the sequence of fluorescence events is a memory driven sequence of events and
(b) it is decided that the fluorescence events observed are due to contaminating nucleotides or other background signals, if the sequence of fluorescence events is not a memory driven sequence of events.

9. Method according to claim 8, characterized in that the catalytic complex comprises a catalyst, a substrate being converted to a product and optionally a cosubstrate.

10. Method according to claim 8 or 9, characterized in that the catalyst is selected from biomolecules, e.g. enzymes, inorganic molecules and organic molecules.

11. Method according to one of the claims 5 -10 wherein an oscillating process is analyzed.
